(19) United States
(12) Patent Application Publication
(10) Pub. No.: US 2013/0011339 A1
(43) Pub. Date: Jan. 10, 2013

(54) NANOPARTICLE COMPOSITIONS COMPRISING A LIPID BILAYER AND ASSOCIATED METHODS

(76) Inventors: Vicki Colvin, Houston, TX (US); Arjun Prakash, Houston, TX (US); Huiguang Zhu, Houston, TX (US)

(21) Appl. No.: 13/346,217

(22) Filed: Jan. 9, 2012

Related U.S. Application Data
(63) Continuation of application No. PCT/US2010/041288, filed on Jul. 8, 2010.
(60) Provisional application No. 61/223,968, filed on Jul. 8, 2009.

(51) Int. Cl. C09K 3/00; A61K 49/18; B05D 7/00; A61K 9/127; A61K 49/00; B82Y 40/00; B82Y 30/00

(52) U.S. Cl. 424/9.321; 252/180; 252/184; 424/450; 424/9.3; 424/9.1; 427/212; 977/773; 977/890

(57) ABSTRACT

Bilayer-nanoparticle compositions comprising a nanoparticle core and a lipid bilayer disposed around the exterior surface of the nanoparticle core are provided. In some embodiments, these bilayer-nanoparticle compositions may be dispersed in an aqueous solution. Associated methods are also provided.

### CROSS REFERENCE TO RELATED APPLICATIONS

This application is a continuation of International Application No. PCT/US2010/41288, filed Jul. 8, 2010, which claims the benefit of U.S. Provisional Application No. 61/223,968, filed Jul. 8, 2009, both of which are incorporated herein by reference.

### STATEMENT OF GOVERNMENT INTEREST

This invention was made with government support under Grant No: EEC-0118007 awarded by the National Science Foundation; Grant No: EEC-0647452 awarded by the National Science Foundation; and Grant No: RD-83253601 awarded by the Environmental Protection Agency. The government has certain rights in the invention.

### BACKGROUND

The present disclosure relates generally to nanoparticle compositions comprising a lipid bilayer and associated methods. In particular, the present disclosure relates to water-soluble nanoparticle compositions comprising a lipid bilayer and associated methods.

The phase transfer of nanoparticles from non-polar to polar suspensions remains an outstanding challenge for material chemists. The best quality nanocrystals, with respect to uniformity, size control and crystallinity, are generally formed in organic solutions at elevated temperatures. These synthetic methods produce nanomaterials as diverse as gold, cadmium selenide and iron oxide which as a consequence of their formation conditions possess surfaces that terminate in organic, non-polar moieties. However, applying the unique optical and magnetic properties of these nanoparticles often requires surface modifications that yield well dispersed and non-aggregated materials stable in water. Nanoscale iron oxides, for example, in water purification as well as magnetic resonance imaging, must be used in aqueous solutions. Quantum dots find enormous application as biological imaging agents, a technology that requires compact and isolated particles whose surfaces are compatible with a variety of biological fluids. These and other uses for nanoparticles have sustained interest in this topic for nearly a decade. Researchers have focused on methods that are both efficient in their transfer of nanoparticles and capable of preventing material aggregation and dissolution.

Many of the existing strategies for nanoparticle phase transfer use lipids as essential components of amphiphilic surface coatings. One commercial quantum dot material reports the use of a proprietary PEG-lipid to create a stable and water soluble material. In these examples, the lipids—typically fatty acids—function as the non-polar constituent of larger amphiphiles (e.g. surfactants). Their hydrophobic tail interacts with the nanocrystal's non-polar organic surface, and leads to a full encapsulation of the core and its original coating. The hydrophilic end of the amphiphile is thus left to stabilize the new surface and renders the material polar and fully dispersed in water. This encapsulation approach ensures that the nanoparticle surfaces are never stripped of their original organic coatings. As a result, particle aggregation is minimized due to the presence of steric stabilization during the entire phase transfer process. Also, in the case of quantum dots, encapsulation is strongly preferred as it prevents degradation of the desirable optical properties. Nanoparticle encapsulation can be problematic in some circumstances as the size of the resulting core and surface treatment can be much larger than the starting organic material. Moreover, it often requires expensive or customized co-polymers and surfactants. Still there are good examples of phase transfer strategies that can produce non-aggregated and stable nanoparticle dispersions in water. These efforts include encapsulation using polymers (e.g., poly-acrylic acid, poly-ethylene glycol) and ligand exchange using moieties such as bifunctional thiols.

Whatever the surface agent selected to affect a phase transfer, it is generally desirable that the resulting nanoparticle suspensions contain little free surfactant or other organic species. Such a criterion is particularly important for biomedical and toxicological studies. Conventional practice relies on sedimentation or filtration to concentrate and purify nanoparticles. These treatments can be intrinsically limited if nanoparticle surface coatings are themselves soluble in water. Unless cross-linked or otherwise irreversibly attached to the nanoparticle, most surface-bound amphiphiles will exist in equilibrium with their free form. As a result, coatings can be removed if nanoparticles are repeatedly washed or diluted. Moreover, the dynamic exchange of the encapsulating agents can result in an adventitious adsorption of other materials, yielding a nanoparticle interface quite different from the one originally engineered. Perhaps the most significant consequence of labile surface coatings is that nanoparticle suspensions must necessarily contain some quantity of the soluble, free surfactant.

These issues motivated our interest in an alternative approach to nanoparticle phase transfer. Our goal was to form small, stable, and non-aggregated nanoparticles in water whose size-dependent properties were preserved; however, we wanted to achieve these features by using a surface coating that in its pure form would have extremely low water solubility.

### SUMMARY

The present disclosure relates generally to nanoparticle compositions comprising a lipid bilayer and associated methods. In particular, the present disclosure relates to water-soluble nanoparticle compositions comprising a lipid bilayer and associated methods.

In one embodiment, the present disclosure provides a composition comprising a nanoparticle core; and a lipid bilayer disposed around the exterior surface of the nanoparticle core.

In another embodiment, the present disclosure provides a composition comprising a bilayer-nanoparticle composition comprising a nanoparticle core and a lipid bilayer disposed around the exterior surface of the nanoparticle core, and an aqueous solution.

In yet another embodiment, the present disclosure provides a method comprising providing a nanoparticle core and one or more fatty acids; and mixing the nanoparticle core with the one or more fatty acids so as to form a bilayer-nanoparticle composition comprising a nanoparticle core and a lipid bilayer disposed around the exterior surface of the nanoparticle core.

The features and advantages of the present disclosure will be readily apparent to those skilled in the art upon a reading of the description of the embodiments that follows.

### DRAWINGS

Some specific example embodiments of the disclosure may be understood by referring, in part, to the following description and the accompanying drawings.

FIG. 1 shows an illustration of the aqueous transfer of iron oxide nanoparticles (nMag) via both (A) addition of IGEPAL® CO 630 surfactant, which generally results in the formation of clusters of nanoparticles in the final aqueous suspensions (B) bilayer formation.

FIGS. 2A and 2B depict thermo-gravimetric analysis (TGA) curves for 10 nm (FIG. 2A) and 17 nm (FIG. 2B) iron oxide nanocrystals. The black dots indicate the percent weight as a function of temperature and the weight loss derivative is indicated in blue. In both the cases, sample mass remained constant while cooling from 900° C. to 50° C.

FIG. 3 shows the variation of the transfer yield of iron oxide (nMAG) nanoparticles and quantum dots (QD) from hexanes into water as a function of oleic acid (OA) concentration. Inset: Scale depiction of a 10 nm diameter nanocrystal coated with a bilayer. Iron oxide concentration was obtained by ICP analysis and quantum dot concentration was obtained via absorbance.

FIGS. 4A and 4B depict the transmission electron micrographs images of iron oxide nanoparticles A) in organics (9.6±1.0 nm), and B) phase transferred into water via bilayer formation (10±1 nm). Inset pictures show phase separated mixtures with water phase at the bottom and hexane phase at the top. As is clear the phase transfer efficiency is on the order of 70% as some color remains in the organic phase. More than 1000 particles were measured to capture both the average size and the size distribution.

FIGS. 5A-5C show small angle X-ray scattering profiles (in black) with simulated fits (in red) for iron oxide nanoparticles in water: A) 10 nm core (bilayer coated), B) 17 nm core (bilayer coated), C) 10 nm core (polymer coated). Inset: corresponding size distributions.

FIG. 6 shows a picture of iron oxide nanocrystal suspensions (10 nm core size) under varying solution phase conditions. Particles that were visibly sedimented or cloudy are surrounded by a red box (designated as precipitated); solutions with unchanged visual appearance are surrounded in green (designated as dispersed). These charge stabilized materials become unstable at low pH, when the fatty acid coatings are protonated (top panel) as well as at high ionic strengths in NaCl (middle panel). Temperature has remarkably little effect on the systems.

FIG. 7 shows the optical and magnetic properties of bilayer-nanoparticle compositions. On the far left panels, a strong permanent magnet is able to concentrate the iron oxide materials (nMAG) much as is observed in hexanes. On the right panel, the fluorescence of quantum dots (QD) is relatively unchanged after the formation of a bilayer and the transfer of the material into water.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the disclosure to the particular forms disclosed, but on the contrary, this disclosure is to cover all modifications and equivalents as illustrated, in part, by the appended claims.

### DESCRIPTION

The present disclosure relates generally to nanoparticle compositions comprising a lipid bilayer and associated methods. In particular, the present disclosure relates to water-soluble nanoparticle compositions comprising a lipid bilayer and associated methods.

The effective water dispersion of highly uniform nanoparticles synthesized in organic solvents is a major issue for their broad applications. The present disclosure provides certain advantages that overcome this problem. Certain embodiments of the present disclosure include the ability to produce nanoparticles surrounded by a lipid bilayer to create stable, aqueous dispersions. Other advantages may also include the ability to transfer a large amount of formed nanoparticles into the aqueous phase. Another advantage includes the production of stable, non-aggregated suspensions comprising nanoparticles that retain their original magnetic and optical properties.

In one embodiment, the present disclosure provides a composition comprising a nanoparticle core and a lipid bilayer disposed around the exterior surface of the nanoparticle core, which may be referred to herein as a “bilayer-nanoparticle composition”. In some embodiments, the use of an exterior lipid bilayer may present a stable and polar interface well suited for a variety of physiological environments. For example, a lipid bilayer disposed around the exterior surface of a nanoparticle may result in the formation of a bilayer-nanoparticle composition that presents polar groups at the particle interface and subsequently leads to particle dispersion in water. In addition, lipid bilayers produced via fatty acids may also have remarkable chemical and thermal stability.

In some embodiments, the methods for producing a bilayer-nanoparticle composition of the present disclosure use the application of a lipid bilayer, which may comprise molecular fatty acids, as a phase transfer agent. In the non-polar nanoparticle solutions, these fatty acids may form dense and compact coatings with the hydrocarbon tail oriented towards the solution phase. If slightly more fatty acid is added in certain embodiments, and the systems appropriately mixed, a second layer of fatty acid may be laid down on top of the original one. This process may result in the formation of a bilayer-nanoparticle composition that presents polar groups at the particle interface and subsequently leads to particle dispersion in water. In some embodiments, the bilayer-nanoparticle composition may have a diameter of about 5 nanometers to about 50 nanometers.

As mentioned above, the bilayer-nanoparticle compositions of the present disclosure generally comprises a nanoparticle core. Examples of suitable nanoparticles may include, but are not limited to, nanoparticles of iron oxide, nickel, nickel-cobalt alloys, cobalt, cobalt alloys, cadmium selenide, noble metal nanoparticles, such as gold and/or silver nanoparticles, and copper nanoparticles. Those of ordinary skill in the art, with the benefit of this disclosure, will be able to select an appropriate nanoparticle for use in embodiments of the present disclosure.

In addition to a nanoparticle core, the bilayer-nanoparticle compositions of the present disclosure also comprise a lipid bilayer. In one embodiment, the lipid bilayer may comprise one or more fatty acids. In some embodiments, both layers of the bilayer are made up of the same moiety. In certain embodiments, the selected fatty acid may be a monomer that is only sparingly soluble in water. In some embodiments, suitable fatty acids may include, but are not limited to, oleic acid, sodium dodecyl benzene sulfonate (“SDBS”), dodecanoic acid, alkanoic acid, and block copolymers of ethylene oxide and propylene oxide, which are commercially available under the trade name PLURONIC® from BASF Chemical Co., and a combination thereof.

The choice of fatty acid may be affected by several factors. For example, the fatty acid may be more effective when its hydrophobic tail is long enough to interact with existing hydrophobic coatings. Similarly, it may be desirable to select a fatty acid that has a poor micelle forming ability, which in some instances may be attributed to the presence of a double bond. Furthermore, in some embodiments, different chain length fatty acids may be utilized to incorporate a bigger size range of particles from about 5-50 nm.

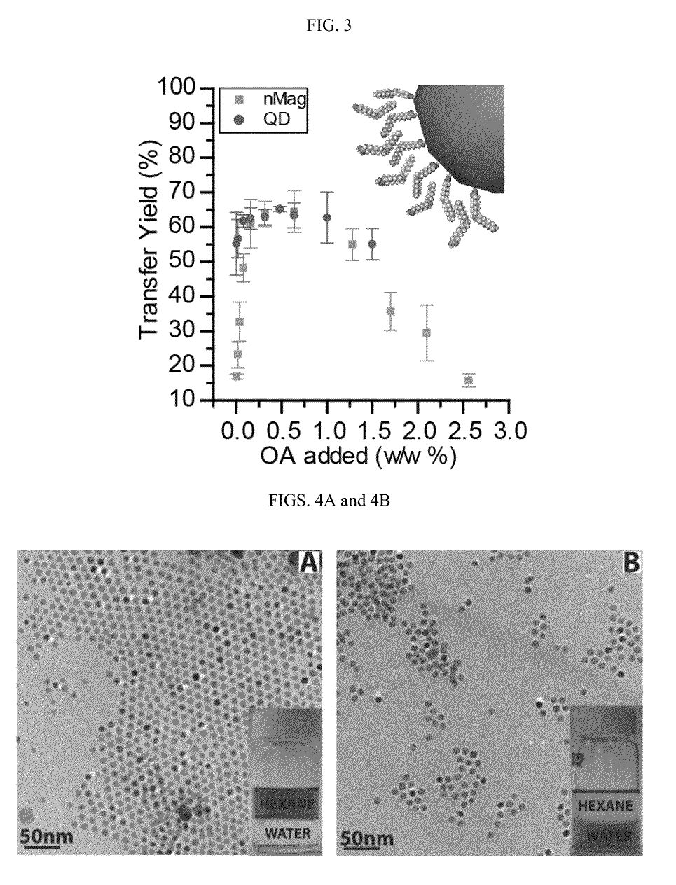

In some embodiments, a lipid bilayer may be formed around a nanoparticle core by the addition of a controlled amount of fatty acid. The formation of a bilayer around the nanoparticle core may be consistent with the strong sensitivity of process yield to fatty acid concentration (FIG. 3). A striking feature of these data is the extremely low quantities of fatty acid required to obtain high phase transfer yields (FIG. 3). In some embodiments, the concentration of the fatty acid should be at or below the critical micelle concentration to ensure bilayer formation. In some embodiments, the amount of fatty acid added may be in the range of from about 0.01 w/w % to about 0.5 w/w %. At higher concentrations, above the critical micelle concentration, the formation of micelles begins to compete with bilayer generation leading to less effective phase transfer. In certain embodiments, where organic solutions were mixed with water and a sparing amount of excess fatty acid, up to about 70% of the nanoparticles were transferred into the aqueous phase.

Unlike other approaches for water dispersion that rely on amphiphiles with significant water solubility, the fatty acids used in the present disclosure are only sparingly soluble in water. As a result, there is minimal dynamic exchange between free and bound surface agents and the resulting aqueous solutions contain little residual free organic carbon. Thermo-gravimetric analysis (TGA) may be used to confirm the presence of a bilayer around a nanoparticle core. The particle size, size distribution, process yield and colloidal stability may also be found using a suite of methods including Transmission Electron Microscopy (TEM), Small Angle X-ray Scattering (SAXS), Dynamic Light Scattering (DLS), Inductively Coupled Plasma-Optical Emission Spectroscopy (ICP-OES) and Ultraviolet-visible Spectroscopy (UV-vis). Bilayer-nanoparticle compositions of the present disclosure possess many of the same size-dependent features as the original materials, and as such offer new avenues for exploring and exploiting the interface between nanoparticles and biology.

While most of the examples provided in this disclosure center on iron oxide nanocrystals, the stabilization of quantum dots has also been explored as illustrated in our study of phase transfer efficiency. The resulting materials possess small hydrodynamic sizes and are stable under a wide range of physiological conditions. SAXS indicates that in contrast to systems stabilized by polymeric surfactants, bilayer-nanoparticle compositions are non-aggregated in water. Little free fatty acid or other organic carbon is measurable in the nanoparticle aqueous suspensions, a result anticipated given the low aqueous solubility of free fatty acid. Bilayer-nanoparticle compositions retain their size dependent physical properties in water.

While the bilayer-nanoparticle compositions of the present disclosure may be useful in numerous applications, they may be particularly useful for water purification, magnetic resonance imaging (MRI), targeted drug delivery, protein separation, and as biological imaging agents.

To facilitate a better understanding of the present disclosure, the following examples of specific embodiments are given. In no way should the following examples be read to limit or define the entire scope of the disclosure.

### EXAMPLES

### Nanocrystal Synthesis

Iron oxy-hydrate (FeO(OH) iron(III) oxide, hydrated; catalyst grade, 30-50 mesh;), 1-octadecene (ODE 90%), cadmium oxide (CdO 99.99%), selenium powder (Se 100 mesh 99%), tri-octylphosphine (TOP 99%), oleic acid (90%) and nitric acid (trace metal grade) were all purchased from SIGMA-ALDRICH®. The 1 μm PTFE AERODISC® syringe filter was purchased from PALL LIFE SCIENCE® and a 0.2 μm NYL syringe filter was purchased from WHATMAN®.

Iron oxide nanoparticles were synthesized by the thermal decomposition of iron carboxylate salts. A mixture of 0.178 g of FeO(OH), 2.26 g oleic acid and 5.0 g of 1-octadecene was stirred and purged with nitrogen; moderate heating up to 280° C. in a three neck-flask led to the formation of an orange solution thought to contain iron carboxylates. Further heating to 320° C. led to a decomposition of this precursor and the generation of brown-black iron oxide nanocrystals. The reaction product was soluble in hexane because of the adsorption of oleic acid to the nanocrystal surface via polar, carboxylate groups.

The iron oxide nanocrystals were purified by repeated cycles of precipitation, sedimentation followed by dispersion in hexane. Reaction products were treated with a 1:1 volumetric amount of acetone and methanol leading to the formation of visible aggregates; these were collected via centrifugation in a pellet and could be dispersed back into hexanes. This procedure was repeated five times to remove unreacted iron salts, 1-ODE or unbound oleic acid. Purified nanocrystal solutions in hexanes could be digested by strong nitric acid and analyzed for their iron content with atomic emission spectroscopy (ICP-OES). Using the average diameter of the material obtained from TEM, and the density of iron oxide (5.17 g/cm³), the atomic concentration of iron could be converted into a nanoparticle concentration.

CdSe nanocrystals were prepared by heating a stirred mixture of 0.3 g of CdO and 2.7 g of oleic acid in a 100 mL three-neck flask at 200° C. until a transparent liquid was obtained. After cooling to room temperature, 15 g of oleic acid and 30 g of ODE was added and the mixture heated to 100° C. under vacuum for 40 minutes. The solution was then purged with ultra-pure N₂ and heated to 300° C. An injection solution, prepared by mixing 10.68 g of Se/TOP (10 wt %) and 4.13 g of ODE, was swiftly injected into the flask with a 20 mL syringe fitted with a large bore needle. After cooling to room temperature, the crude quantum dots were precipitated by the addition of acetone and methanol, in a fashion similar to that used for the iron oxide. These pellets could be isolated and purified after repeated centrifugation at 3500 g followed by redispersion into hexanes. The final purified nanocrystal pellet was ultimately redispersed in hexane, filtered through 1 μm PTFE syringe filter and stored in the dark. Quantum dot concentrations were estimated from absorbance data using methods published elsewhere.

Phase Transfer of Nanocrystals:

Oleic acid was added in variable amounts (0.5-300 μL) to 1.0 mL of purified nanocrystal suspensions in hexanes (typically 1 g nanoparticle/L). The resulting solution was then sonicated in a bath for one minute with no visible change in appearance (FS6 sonicator from FISCHER SCIENTIFIC®). Next, 10 mL of ultrapure water (MILLIPORE®, 18.2MΩcm) was then added to the hexane solution resulting in an obvious phase separation between the clear water and colored non-polar solution. To affect the transfer of material from hexanes into water, this solution was subjected to sonication via a probe (UP 50H probe sonciator from DR.HIELSCHER®) for 5 minutes at 50% amplitude and full cycle. Care was taken to ensure that the tip of the probe, where the power is the highest, was located near the interfacial region between the hexanes and water phase. Immediately after sonication a cloudy and colored solution was obtained, but if left to sit undisturbed for one day the mixture phase separated with the colored nanoparticles appearing in the bottom, aqueous fraction. This layer was collected and the nanoparticles purified via centrifugation at 3500 g for 15 min, followed by redispersion and filtration through a syringe filter (pore size of 0.2 μm, Whatman-NYL). The filtered product was a clear, colored suspension that could be further concentrated (typically 10×) via rotary evaporation. The above procedure was also used to phase transfer cadmium selenide nanocrystals. Methods to describe the phase transfer using IGEPAL® CO 630 are described elsewhere.

Characterization of Nanocrystals:

Small Angle X-ray Scattering (SAXS) profiles were obtained on a RIGAKU SMARTLAB® system operating in transmission mode with a line collimation setup. A capillary tube (0.8 mm diameter) was filled with a sample, and the low angle scattering was collected from 2θ values of 0.15 to 4 degrees with a Cu K-α X-ray beam of wavelength 1.54 Å. The X-rays were generated at 40 kv and 44 mA. Typical data collection times were on the order of two hours. The raw scattering data was analyzed using Rigaku's NANOSOLVER® software using a split interval of 30 with low slit correction factor.

Dynamic Light Scattering (DLS) data was collected using a BROOKHAVEN® instrument equipped with a BI-9000AT digital autocorrelator using a monitoring wavelength of 656 nm. Standard 1.5 mL poly-methacrylate cuvettes were used as sample holders and each sample was analyzed for 4 minutes to obtain a minimum intensity of 200,000 cps. A histogram of the particle diameter distribution was obtained via a “Contin” fit to the raw autocorrelation data.

Transmission electron microscopy (TEM) was carried out using JEOL 2100 field emission gun TEM at 200 kV with a single tilt holder using 300 mesh copper grids with holey carbon from Ted Pella Inc. Thermo-gravitmetric Analysis (TGA) was carried out using TA INSTRUMENTS® SDT 2960 simultaneous DSC-TGA instrument with sample deposited in a platinum pan. Samples were maintained at 150° C. for 5 hrs for removal of any associated solvent/moisture before further heating. The samples were then heated to 900° C. at the rate of 50° C./min. UV-visible spectroscopy was carried out using Cary 5000 VARIAN® UV-vis-NIR spectrophotometer with 1.5 mL poly-methacrylate cuvettes used as sample holders. All sizing data with respective significant figures was reported with error bars representing their standard deviation. Inductively coupled plasma (ICP) analysis was carried out using PERKIN ELMER® ICP-OES instrument equipped with auto-sampler. Total organic carbon content of the supernatant was computed using SHIMADZU® TOC analyzer after sedimentation of particle suspension (1 mL of 1 μM) (BECKMAN-COULTER OPTIMA® L-80XP Ultracentrifuge) at 118,000 g for 4 hours at 25° C.

Results:

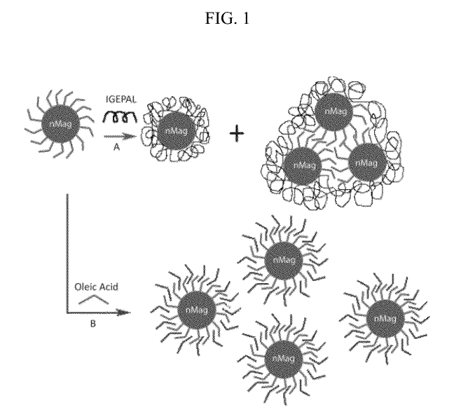

This disclosure focuses on creating stable aqueous suspensions of bilayer-nanoparticle compositions. FIG. 1B illustrates one embodiment of the present disclosure wherein a bilayer-nanoparticle composition is formed through the use of oleic acid and iron oxide nanoparticles (nMag) and contrasts it to the more conventional use of polymeric surfactants (FIG. 1A) to affect nanoparticle phase transfer. While only one method forms a bilayer, both methods use amphiphiles to change the interfacial chemistry of particles from non-polar to polar.

To explore the present disclosure, oleic acid—a C18 unsaturated fatty acid—was added as a phase transfer agent to an oil/water mixture of iron oxide or cadmium selenide nanoparticles. Under the appropriate mixing conditions, for example probe sonication, the colored nanocrystals were transferred from the organic to aqueous layers with high efficiency. In certain embodiments the colored nanocrystals were transferred from the organic to the aqueous layer with an efficiency of about 70%. Several characterization methods were applied to confirm that the surfaces of the nanoparticles in water were covered in bilayers; among these thermo-gravimetric analysis (TGA) was the most conclusive for these structures.

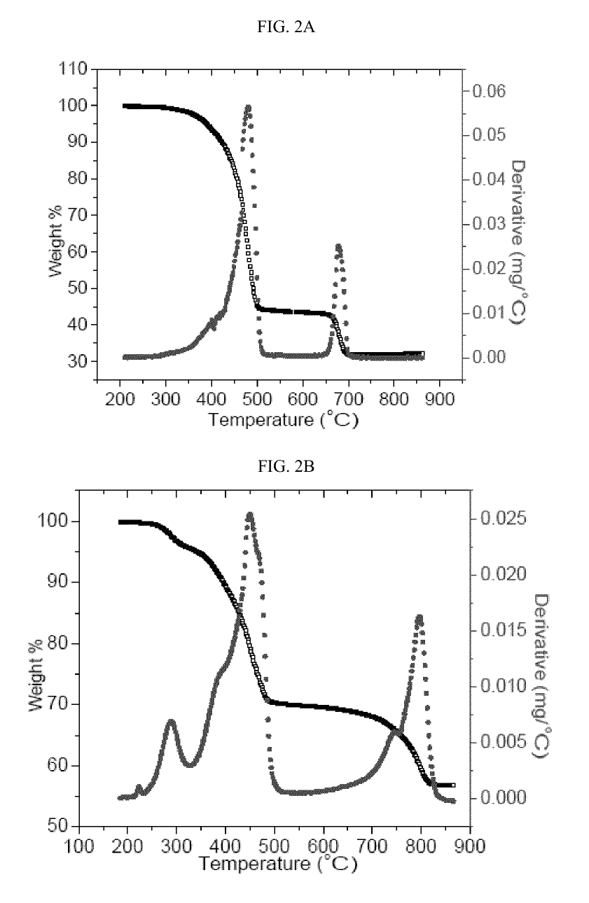

During controlled heating of sample residues, two distinct weight loss peaks were observed. These correlate well in temperature to those reported for fatty acid double layers in a variety of environments (FIG. 2). The mass loss between 400 to 500° C. corresponds to the desorption of the outer layer of oleic acid; as expected, it occurs at a temperature slightly higher than the boiling point of neat oleic acid or 360° C. at 760 mm Hg. A second inflection point occurs between 650 and 800° C. This feature arises from the loss of more tightly bound oleic acid. This inner layer of oleic acid is thought to be stabilized via a complex between iron (II) and the carboxylate groups of oleic acid. As a result, it can only be removed from the surface at higher temperatures. Also, it is noted that the weight loss difference between the outer and inner layers can be semi-quantitatively attributed to the higher curvature of the smaller 10 nm particle in comparison with the bigger 17 nm particle. The coincidence of the TGA peak temperatures in these samples with that reported previously for bilayers, both on surfaces and colloids, is strong evidence that the materials are stabilized by oleic acid bilayers.

For both quantum dots and iron oxide nanocrystals, over 70% of the material was transferred from hexanes to water after the addition of only 0.2 w/w % oleic acid. This is in stark contrast to particle stabilization with IGEPAL® CO 630 which requires more than 10 w/w % for reasonable phase transfer yields. This observation is likely due to the competition between oleic acid micelle and bilayer formation. At or near its critical micelle concentration (CMC), oleic acid can form micelles in water and this process would remove bilayer material from the surface and reduce the solubility in water. As a result, the optimal phase transfer efficiency is obtained near the CMC for oleic acid. This observation may explain why reports of bilayer phase transfer methods for highly uniform nanocrystals are limited: conventional practice involves the addition of a vast excess of phase transfer agent to a suspension in order to ensure an efficient process. As apparent in FIG. 3, such an approach would depress the transfer efficiencies substantially. In general, if bilayer formation is desired, it is best to work with fatty acid concentrations (0.7 to 3.5 mM for oleic acid) that are at or below the critical micelle concentration. At their highest transfer yields, the molar ratio of oleic acid to nanoparticles was found to be 90 for 10 nm iron oxide and 17 for 4 nm quantum dots. This observation between the two nanoparticle systems could be attributed to the order of magnitude difference in their surface areas.

Also notable in FIG. 3 is the similarity between the process yield for both quantum dots and iron oxide nanocrystals. Not only is the core composition different in these two cases, but the core diameters are also very different (e.g. 4 nm diameter as opposed to 10 nm diameter). Still the behavior and optimization is comparable suggesting that as long as particles possess a hydrophobic surface, the addition of small amounts of fatty acid may be suitable for creating water stable dispersions.

The total organic carbon (TOC) found free in solution for the oleic acid stabilized nanoparticles is just 9 ppm—three orders of magnitude less than that found for equivalent polymer encapsulated (IGEPAL® CO 630) materials. This observation can be explained by the different solubilities of oleic acid versus conventional phase transfer agents. Large amounts of polymeric surfactants like IGEPAL® are required to affect nanoparticle phase separation because these materials alone have high solubility in water. An excess of free polymer in the aqueous suspensions ensures a complete and stable surface coating. In contrast, oleic acid is virtually insoluble in water (HLB value of 1) and once incorporated into a bilayer structure will not appreciably desorb from the surface. The price paid for an insoluble surface stabilizing agent is the challenge associated with combining the original hydrophobic nanoparticles, free oleic acids, and water. Here this kinetic barrier is overcome by using a brief ultrasonication process which quickly mixes the various components and results in stable aqueous suspensions. While our phase transfer yields are quite high—on the order of 70%—they are not 100% effective and this is likely due to the challenges of mixing the disparate starting materials (FIG. 3). It may also be possible to replace ultrasonication with elevated temperatures for more polar fatty acids and their salts.

An important concern for applications of nanocrystals in water is that the phase transfer process should preserve the original quality of the material as well as prevent particle aggregation. The first issue is of particular concern in this process as it relies on probe sonication to ensure adequate mixing of the insoluble fatty acids, nanocrystals and water. The preparation of nanocrystals in organic media affords a great deal of control over nanocrystal nucleation and growth, and as a result the as-synthesized nanocrystals possess symmetric shapes, narrow size distributions, and high crystallinity (FIG. 4A). These desirable features remain unchanged after the phase transfer process (FIG. 4B). Most notably, each particle is well separated from its neighbors in the microscopy images, suggesting that an organic coating is associated with individual particles. Particle aggregation is not prevalent in the dried films. This observation is supported by the high clarity of the suspensions and their apparent lack of sedimentation over months (insets FIG. 4).

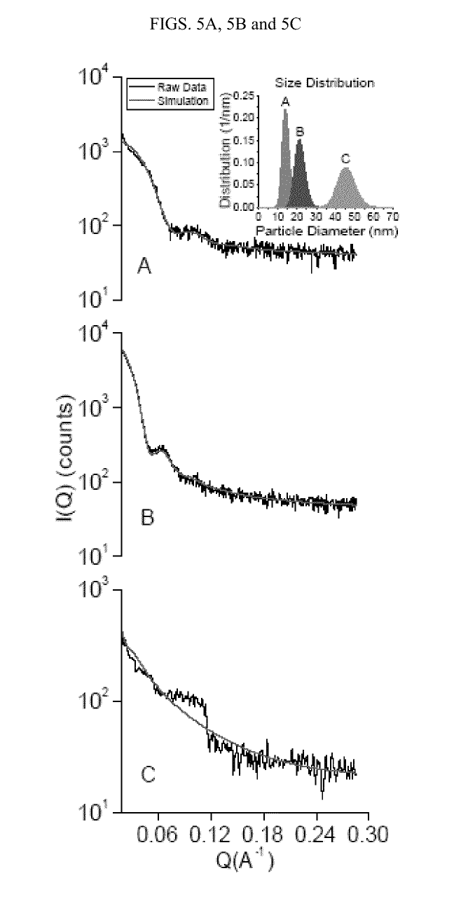

Direct evidence that bilayer-nanoparticle compositions are not aggregated is found in an analysis of their small angle X-ray scattering (SAXS) profiles. This method is sensitive to the presence of aggregates from two to ten particles across, and complements well the visual observations and microscopic analysis in FIG. 4. FIGS. 5A and 5B present the SAXS profiles for bilayer coated iron oxide nanocrystals of 10 and 17 nm core diameters in water. The inverted scattering minima at low angles are very sensitive to the particle size distribution, and their appearance in the raw data confirm the finding from TEM that these are highly uniform samples. Models for X-ray scattering of small particles can be fit to these data to obtain more quantitative information, and these take as inputs the density, expected size and size distribution of the particles. The best fits to the scattering data are shown as solid lines and the resulting overall size distributions are shown in the figure inset. As expected, the larger core sizes lead to greater diameters; moreover, the average sizes are representative of non-aggregated and fully isolated nanoparticles. These basic conclusions were confirmed by dynamic light scattering data (Table 1) which provides a semi-quantitative measurement of the average hydrodynamic diameter of nanocrystals directly in suspension.

The hydrodynamic diameters of these materials are in good agreement with what is expected for an inorganic core surrounded by a fatty acid bilayer (Table 1). In this analysis, dimensions found from multiple characterization methods were compared to extract the effective thickness of the bilayers. TEM provides the inorganic core diameter; SAXS analysis provides a measure of the extent of the core and the dense organic coatings; and finally, DLS data reports the full hydrodynamic diameter of the bilayer-nanocrystal complex and associated hydration shell. The diameters obtained via SAXS and DLS for bilayer-nanocrystal complexes (Sample A and Sample B) are larger than the inorganic core as expected; the 4.6 nanometer difference (average of Samples A and B shell size) between the nanoparticle cores and the bilayer-nanoparticle composition can be attributed to the oleic acid bilayer. This corresponds to a surface coating thickness of about 2.3 nm which is comparable to the thickness of C-18 chain bilayers measured in other similar systems. Both in these systems, as well in other oleic acid bilayers, there is a large degree of interpenetration of the C-18 chains—a feature depicted schematically in FIG. 3. Sample B corresponds to 17 nm core iron oxide particles coated with oleic acid bilayer. Similar bilayer dimensions were obtained via SAXS and DLS for this case and these are consistent with that of the smaller sample A.

TABLE 1

| | Sample | | |
|---|---|---|---|
| | A (nm) | B (nm) | C (nm) |
| TEM (core) | 10.0 ± 1.2 | 16.6 ± 2.3 | 10.0 ± 1.2 |
| SAXS (core + shell) | 14.3 ± 1.8 | 21.5 ± 2.6 | 49.0 ± 4.8 |
| DLS (hydrodynamic) | 14.2 ± 2.6 | 26.3 ± 4.1 | 154.1 ± 15.6 |

An important feature of bilayer-nanocrystal complexes highlighted by Table 1 is that they form compact structures in aqueous suspensions. Typically, the bilayer-nanocrystal complexes are only 4.6 nm larger than the core nanocrystal. Quantum dots stabilized by amphiphilic surfactants, for example, can possess hydrodynamic diameters nearly five to ten times larger than their core diameter.

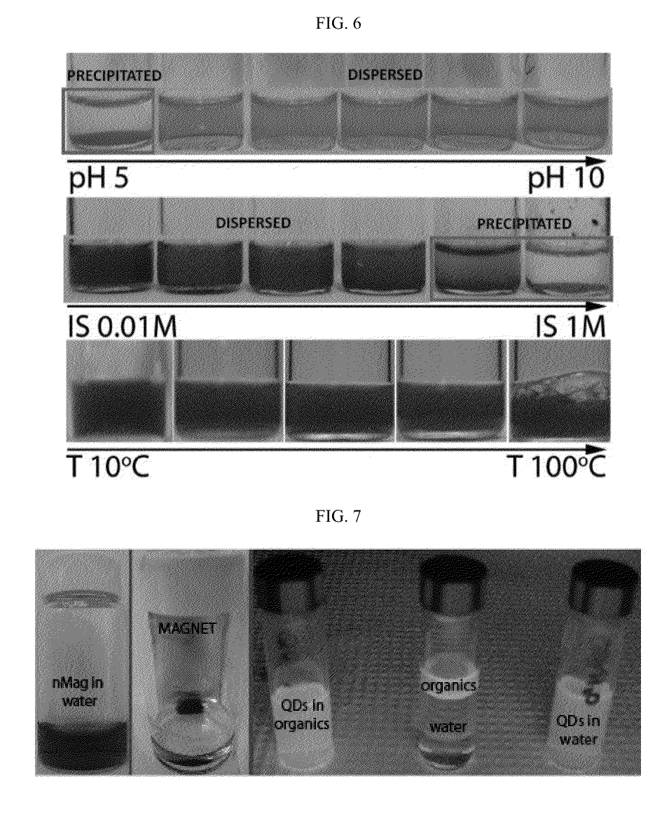

Bilayers produced via fatty acids, such as oleic acid, have remarkable chemical and thermal stability. The formation of a bilayer on the nanoparticle surface leads to a pH dependent charge stabilization confirmed by Zeta potential measurements (−55 mV at a pH of 6.0). FIG. 6 shows the visual appearance of bilayer-nanocrystal complex suspensions under different conditions of pH, ionic strength and temperature. As expected for these systems, in acidic conditions the surface groups are protonated; above the pKa of oleic acid (≈5.0), however, the nanocrystals are quite stable. The addition of salts to these suspensions can result in the precipitation of nanocrystal aggregates; the middle panel of FIG. 6 illustrates that above 250 mM the electrostatic repulsion is effectively shielded and interparticle aggregation becomes substantial. DLS confirms these visual observations. While the materials are somewhat sensitive to both charge and pH, they are remarkably stable over a variety of temperatures (bottom, FIG. 6).

A delineation of the unique and valuable optical and magnetic properties of nanoparticles has been the subject of extensive prior work. Here, it is confirmed that the important physical properties of the bilayer-nanoparticle compositions remain unchanged after transfer into water (FIG. 7). Nanocrystalline iron oxide phase transferred into water can be captured by an external magnetic field; the time for capture and the overall efficiency of the process is unchanged as would be expected given the physical characterization of the materials (FIG. 4). FIG. 7 also illustrates that the optical properties of quantum dots before and after bilayer stabilization are relatively unchanged. Most importantly, the quantum yield for these quantum dots systems remains within 20% of its original value after phase transfer.

Finally, in order to identify the advantages of a bilayer stabilization approach, these results were compared to those found using a conventional polymeric surfactant, IGEPAL® CO 630. FIG. 5C shows that these surfactants when applied to iron oxide nanocrystals result in particle aggregation; larger amphiphilic phase transfer agents have been reported to encapsulate multiple particles. This results in polydisperse groupings of iron oxide nanocrystals. Also, corresponding DLS diameters (Table 1: sample C) show a significant increase over the particle core size, indicating the presence of aggregates in polymer stabilized materials. These observations highlight the significant challenges faced in preventing aggregation of these magnetic materials during phase transfer. The approach outlined in this disclosure, in contrast, is successful in generating isolated magnetic nanocrystals as well as quantum dots.

Therefore, the present disclosure is well adapted to attain the ends and advantages mentioned as well as those that are inherent therein. While numerous changes may be made by those skilled in the art, such changes are encompassed within the spirit of this disclosure as illustrated, in part, by the appended claims.

What is claimed is:

1. A composition comprising:
a nanoparticle core; and
a lipid bilayer disposed around the exterior surface of the nanoparticle core.

2. The composition of claim 1 where the lipid bilayer comprises one or more fatty acids.

3. The composition of claim 2 wherein the fatty acid comprises one or more fatty acids selected from the group consisting of: oleic acid, sodium dodecyl benzene sulfonate, dodecanoic acid, alkanoic acid, block copolymers of ethylene oxide and propylene oxide, and a combination thereof.

4. The composition of claim 2 wherein the fatty acid comprises oleic acid.

5. The composition of claim 1 further comprising an aqueous solution.

6. The composition of claim 1 wherein the nanoparticle core comprises one or more materials selected from the group consisting of: iron oxide, nickel, nickel-cobalt alloys, cobalt, cobalt alloys, cadmium selenide, gold, silver, copper, and a combination thereof.

7. The composition of claim 1 wherein the diameter is from about 5 to about 50 nm.

8. A composition comprising:
a bilayer-nanoparticle composition comprising a nanoparticle core and a lipid bilayer disposed around the exterior surface of the nanoparticle core, and
an aqueous solution.

9. The composition of claim 8 where the lipid bilayer comprises one or more fatty acids selected from the group consisting of: oleic acid, sodium dodecyl benzene sulfonate, dodecanoic acid, alkanoic acid, block copolymers of ethylene oxide and propylene oxide, and a combination thereof.

10. The composition of claim 9 wherein the fatty acid comprises oleic acid.

11. The composition of claim 8 wherein the nanoparticle core comprises one or more materials selected from the group consisting of: iron oxide, nickel, nickel-cobalt alloys, cobalt, cobalt alloys, cadmium selenide, gold, silver, copper, and a combination thereof.

12. A method comprising:
providing a nanoparticle core and one or more fatty acids; and
mixing the nanoparticle core with the one or more fatty acids so as to form a bilayer-nanoparticle composition comprising a nanoparticle core and a lipid bilayer disposed around the exterior surface of the nanoparticle core.

13. The method of claim 12 further comprising mixing the bilayer-nanoparticle composition with an aqueous solution.

14. The method of claim 13 wherein the aqueous solution is water.

15. The method of claim 13 wherein the bilayer-nanoparticle composition are transferred to the aqueous phase in an amount up to about 70%.

16. The method of claim 13 wherein the fatty acid comprises one or more fatty acids selected from the group consisting of: oleic acid, sodium dodecyl benzene sulfonate, dodecanoic acid, alkanoic acid, block copolymers of ethylene oxide and propylene oxide, and a combination thereof.

17. The method of claim 13 wherein the nanoparticle core comprises one or more materials selected from the group consisting of: iron oxide, nickel, nickel-cobalt alloys, cobalt, cobalt alloys, cadmium selenide, gold, silver, copper, and a combination thereof.

18. The method of claim 13 wherein the bilayer-nanoparticle composition has a diameter from about 5 to about 50 nm.

19. The method of claim 13 wherein the fatty acid is present in an mount of about 0.01 w/w % to about 0.5 w/w %.

20. The method of claim 13 wherein a plurality of non-aggregated bilayer-nanoparticle compositions are formed.
